(19) United States
(12) Patent Application Publication
(10) Pub. No.: US 2017/0189109 A1
(43) Pub. Date: Jul. 6, 2017

(54) ELECTROSURGICAL APPARATUS

(71) Applicant: GYRUS MEDICAL LIMITED, Cardiff (GB)

(72) Inventor: Kelvin John VARNEY, Abergavenny (GB)

(73) Assignee: GYRUS MEDICAL LIMITED, Cardiff (GB)

(21) Appl. No.: 15/400,352

(22) Filed: Jan. 6, 2017

(30) Foreign Application Priority Data
Jan. 6, 2016 (GB) 1600202.4

(51) Int. Cl. A61B 18/14; A61B 18/04; A61B 18/16

(52) U.S. Cl. CPC A61B 18/1492; A61B 18/16; A61B 18/042; A61B 2018/00589

(57) ABSTRACT

An electrosurgical apparatus for coagulating tissue comprises an elongated tube (6) having a tubular wall (13) and a proximal end and a distal end, and an elongate sheath (3) into which the tube (6) is inserted. The tube constites a conduit (11) though which ionisable gas can be supplied to the distal end of the tube. The tube includes one or more apertures (20) in the tube such that the ionisable gas is capable of exiting the tube in the region of the distal end of the tube. An intermediate sleeve (14) is provided into which the elongated tube (6) is inserted, and a tube handle (17) is attached to the proximal end of the tube. A sleeve handle (18) prevents the sleeve (14) from rotating, such that actuation of the handle (17) causes the flexible elongated tube to rotate within the intermediate sleeve (14).

### TECHNICAL FIELD

Embodiments of the invention relates to an electrosurgical apparatus and in particular to the non-contact coagulation of tissue using an ionisable gas such as argon.

### BACKGROUND TO THE INVENTION AND PRIOR ART

Argon beam coagulators have been known for many years, and examples are given in U.S. Pat. Nos. 4,040,426, 5,720,745, 6,039,736 and 6,197,026. The first example is an end-effect instrument, in which the ionised gas exits through the end of the instrument, while the latter two examples are directed at side-effect instruments, in which the ionised gas exits the instrument though an aperture in the side of the instrument. Such instruments are often referred to as APC instruments (Argon Plasma Coagulation).

APC instruments are often used in endoluminal procedures in which the length of the instrument is many times its diameter. In such situations it is often difficult to manipulate the tip of the instrument precisely into position, as a movement instigated at the proximal end of the instrument has a large distance to travel before constituting a corresponding movement at the distal end of the instrument.

### SUMMARY OF THE INVENTION

Embodiments of the invention attempt to provide an instrument which is more versatile than any of the instruments in the prior art, and accordingly some examples provide an electrosurgical apparatus for coagulating tissue, comprising a flexible elongated tube having a proximal end and a distal end, an elongate sheath into which the flexible tube is inserted, a conduit though which ionisable gas can be supplied to the distal end of the tube, the tube including one or more apertures in the tube such that the ionisable gas is capable of exiting the tube in the region of the distal end of the tube, at least one electrode for ionising the ionisable gas prior to the gas exiting the one or more apertures, an intermediate sleeve into which the flexible elongated tube is inserted, a tube handle attached to the proximal end of the flexible elongated tube, and means for preventing the flexible sleeve from rotating, such that actuation of the handle causes the flexible elongated tube to rotate within the intermediate sleeve.

The intermediate sleeve ensures that rotation or longitudinal movement of the proximal end of the elongate tube is translated into a corresponding movement of the distal end of the tube, without the application of excessive force. Accordingly, the intermediate sleeve is conveniently formed of a low friction material.

As mentioned previously, the length of the tube is often many times its diameter. Therefore, the intermediate sleeve conveniently has a length more than 10 times its diameter, preferably more than 50 times its diameter, typically more than 100 times its diameter, and conceivably more than 500 times its diameter. According to one convenient arrangement, the intermediate sleeve extends the majority of the length of the elongated tube. Preferably, the intermediate sleeve extends to the distal end of the elongate tube with the exception of a distal end portion, the distal end portion containing the one or more apertures. In this way, the elongate tube is in contact with the intermediate sleeve along the majority if not the near entirety of its length. This provides an easier environment for movement of the tube as opposed to it being in contact with the bore of an endoscope, as would be the case in prior art instruments.

Preferably, one or both of the intermediate sleeve and the elongated tube is provided with friction reducing features. The friction reducing features are conveniently a plurality of projections, typically elongate projections. The friction reducing features are preferably present on the intermediate sleeve, although they can of course alternatively be provided on the tube itself. Typical projections are continuous raised ridges, so as to provide a smaller area of contact between the tube and the sleeve, hence reducing the frictional drag therebetween. The projections are conceivably longitudinal, helical, or intermittent, and can be of any cross-section effective to provide a low friction contact between the tube and the sleeve.

The means for preventing the intermediate sleeve from rotating conveniently comprises a sleeve handle capable of being held to prevent rotation of the intermediate sleeve. Thus the user of the APC instrument moves the elongate tube using the tube handle, while holding the sleeve handle in order to maintain the sleeve stationary.

The intermediate sleeve is conveniently formed of PTFE. Conceivably, the PTFE sleeve has a wire embedded therein, typically wound in a helix within the PTFE material of the sleeve, or alternatively in the form of a braid embedded within the PTFE material of the sleeve. The wire serves to stiffen the sleeve to prevent it from becoming twisted as the tube is rotated. Conceivably, the wire can have a dual purpose, being used to create the projections forming the friction reducing features as described previously.

### DESCRIPTION OF THE DRAWINGS

Embodiments of the invention will now be further described, by way of example only, with reference to the accompanying drawings, in which:

FIG. 1 is a schematic view of an electrosurgical system employing an instrument in accordance with an embodiment of the present invention,

FIG. 2 is a schematic part-sectional view of the proximal portion of an electrosurgical instrument in accordance with an embodiment of the present invention,

FIG. 3 is a schematic part-sectional view of an intermediate portion of an electrosurgical instrument in accordance with an embodiment of the present invention,

FIG. 4 is a schematic perspective view of a distal portion of an electrosurgical instrument according to an embodiment of the invention,

FIG. 5 is a cross-sectional view of the electrosurgical instrument of FIG. 2,

FIG. 6 is a cross-sectional view of an alternative embodiment of electrosurgical instrument according to an embodiment of the invention,

FIG. 7 is a cross-sectional view of a further embodiment of electrosurgical instrument according to an embodiment of the invention,

FIG. 8 is a sectional side view of a part of the electrosurgical instrument of FIG. 7,

FIG. 9 is a cross-sectional view of a further embodiment of electrosurgical instrument according to an embodiment of the invention, and

FIG. 10 is a sectional view of a distal portion of an alternative embodiment of electrosurgical instrument according to an embodiment of the invention.

### DESCRIPTION OF THE EMBODIMENTS

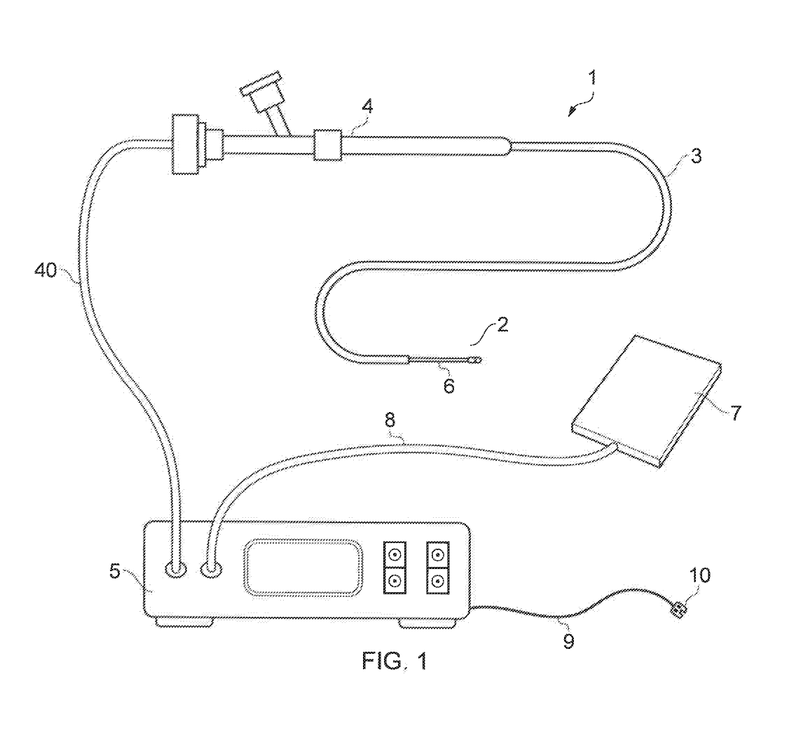

Referring to FIG. 1, an APC system comprises an instrument shown generally at 1, and comprising a working instrument 2 disposed through an elongate flexible sheath 3 extending from an endoscope 4. The working instrument 2 is connected to a radio frequency generator 5 via flexible cable 40, the generator 5 also including a source of argon gas (not shown) which is also supplied through the cable 40. The working instrument 2 comprises an elongate tube 6 to be described in more detail subsequently. A patient return plate 7 is also connected to the generator 5 by means of cable 8. The generator 5 is connected to a source of power by lead 9 and plug 10.

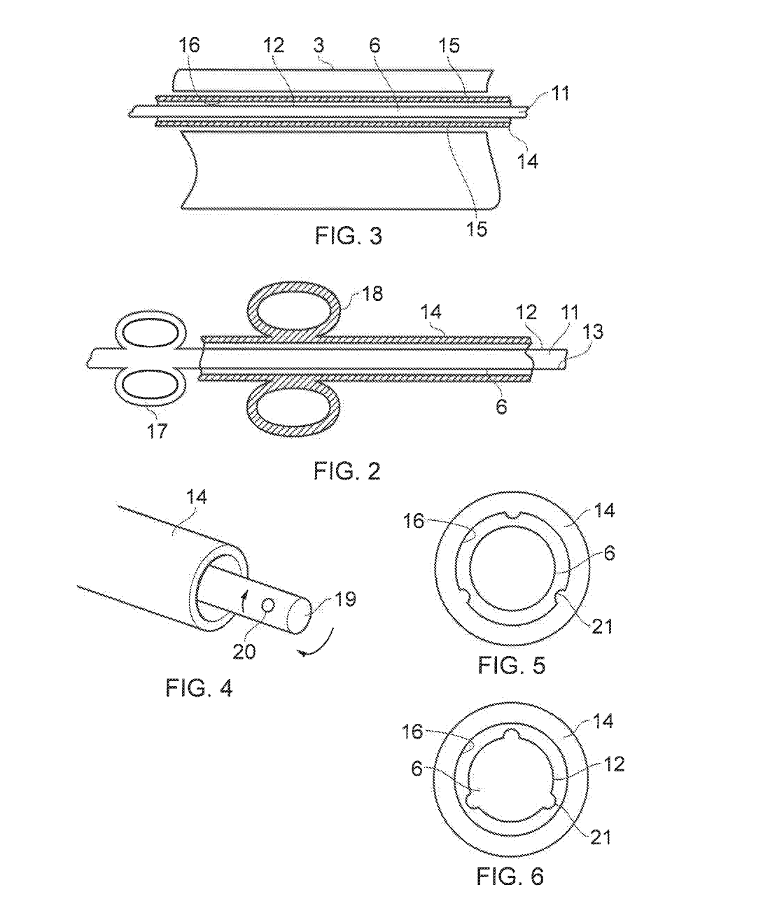

FIGS. 2 to 4 show the proximal, intermediate and distal parts of the working instrument 2. The elongate tube 6 is hollow so as to form a gas conduit 11 therein, and includes an outer wall 12 and an inner wall 13. The tube 6 is formed of an electrically insulating material such as a durable plastics material. An intermediate sleeve 14 of PTFE material is located between the sheath 3 and the elongate tube 6. The sleeve 14 has an outer surface 15 and an inner surface 16. The tube 6 is located within the sleeve 14 such that the outer wall 12 of the tube is in contact with the inner surface 16 of the sleeve rather than the sheath 3.

A tube handle 17 is connected to the proximal end of the tube 6 as shown in FIG. 2, and a sleeve handle 18 is connected to the sleeve 14 near its proximal end. Relative movement between the tube 6 and the sleeve 14 can be effected by moving handle 17 relative to handle 18, whether it be a longitudinal movement, a rotational movement or a combination of the two.

FIG. 4 shows the distal end of the tube 6, which has an end face 19 effectively blanking off the end of the tube, and a side aperture 20 through which an ionisable gas introduced through the gas conduit 11 exits the tube. In use, radio frequency energy is provided from the generator 5 to an electrode (not shown) within the tube such that the gas is ionised into a plasma when it exits the aperture 20. This ionised gas impinges upon tissue adjacent the aperture in order to coagulate it, in a manner well known in the art.

FIG. 5 shows a version of the intermediate sleeve 14 containing friction reducing features in the form of elongate projections 21 present on the inner surface 16 of the sleeve. The projections run the length of the sleeve and form the contact areas between the tube 6 and the sleeve 14. As the tube contacts only the projections 21 and not the entire inner surface 16 of the sleeve, the contact area is much reduced. This reduced contact area provides a much lower frictional drag between the tube 6 and the sleeve 14, allowing easier movement of the tube within the sleeve. The projections 21 may be continuous or intermittent, straight or curved, and a helical “rifled” arrangement is particularly effective. FIG. 6 shows an alternative arrangement in which the projections 21 are provided on the outer wall 12 of the tube 6 rather than inner surface 16 of the sleeve 14.

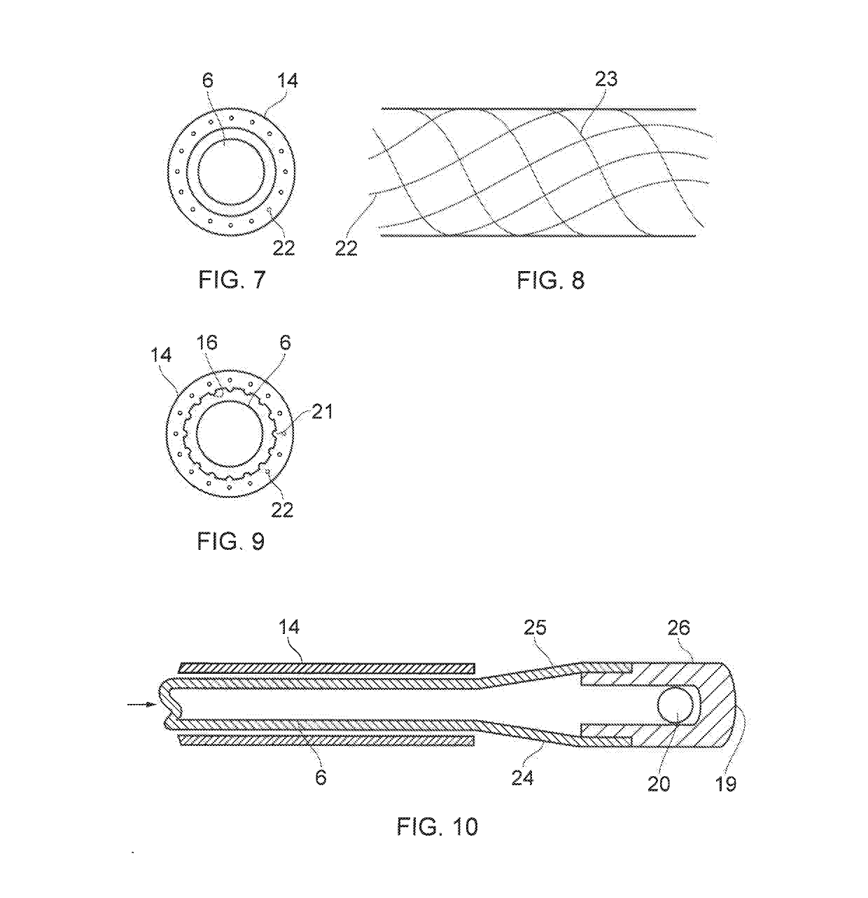

FIGS. 7 & 8 show a further alternative in which the intermediate sleeve 14 is provided with wires 22 embedded therein. The wires 22 serve to increase the stiffness of the sleeve 14, and resist any twisting of the sleeve due to the frictional drag thereon when the tube 6 is rotated. The wires 22 are in the form of a helical braid, as shown at 23 in FIG. 8. FIG. 9 shows an alternative embodiment in which at least some of the wires 22 are located close to the inner surface 16 of the sleeve. In this way, those wires which are close to the surface create the projections 21 that constitute the friction reducing features.

Finally, FIG. 10 shows the distal tip of the tube 6 in an alternative embodiment of the invention. The tube 6 is contained within the intermediate sleeve 14 until its distal portion 24, at which it extends from the sleeve and flares outwardly as shown at 25. An end-piece 26 is fitted into the end of the tube 6, and includes the end face 19 and side aperture 20. As before, the electrode ionising the gas within the tube is not shown for clarity.

Those skilled in the art will appreciate that other constructions can be envisaged without departing from the scope of the present invention. For example, the number, location and shape of the apertures can be varied, as can the shape of the electrode element. Monopolar or bipolar versions of the instrument are possible, depending on the intended use, and different versions of the system can be envisaged for different endoscopic procedures.

1. An electrosurgical apparatus for coagulating tissue, comprising:
a flexible elongated tube having a proximal end and a distal end,
an elongate sheath into which the flexible tube is inserted,
a conduit though which ionisable gas can be supplied to the distal end of the tube, the tube including one or more apertures in the tube such that the ionisable gas is capable of exiting the tube in the region of the distal end of the tube,
at least one electrode for ionising the ionisable gas prior to the gas exiting the one or more apertures,
an intermediate sleeve situated between the tube and the elongate sheath,
a tube handle attached to the proximal end of the flexible elongated tube, and
means for preventing the intermediate sleeve from rotating, such that actuation of the handle causes the flexible elongated tube to rotate within the intermediate sleeve.

2. Apparatus according to claim 1, wherein the intermediate sleeve is formed of a low friction material.

3. Apparatus according to claim 1, wherein the intermediate sleeve has a length more than 10 times its diameter.

4. Apparatus according to claim 3, wherein the intermediate sleeve has a length more than 50 times its diameter.

5. Apparatus according to claim 4, wherein the intermediate sleeve has a length more than 100 times its diameter.

6. Apparatus according to claim 5, wherein the intermediate sleeve has a length more than 500 times its diameter.

7. Apparatus according to claim 1, wherein the intermediate sleeve extends the majority of the length of the elongated tube.

8. Apparatus according to claim 7, wherein the intermediate sleeve extends to the distal end of the elongate tube with the exception of a distal end portion, the distal end portion containing the one or more apertures.

9. Apparatus according to claim 1, wherein one or both of the intermediate sleeve and the elongated tube is provided with friction reducing features.

10. Apparatus according to claim 9, wherein the friction reducing features are a plurality of projections.

11. Apparatus according to claim 10, wherein the friction reducing features are a plurality of elongate projections.

12. Apparatus according to claim 9, wherein the friction reducing features are present on the intermediate sleeve.

13. Apparatus according to claim 1, wherein the means for preventing the intermediate sleeve from rotating comprises a sleeve handle capable of being held to prevent rotation of the intermediate sleeve.

14. Apparatus according to claim 1, wherein the intermediate sleeve is formed of PTFE.

15. Apparatus according to claim 1, wherein the intermediate sleeve has a wire embedded therein.

16. Apparatus according to claim 15, wherein the wire is wound in a helix within the material of the sleeve.

17. Apparatus according to claim 15, wherein the wire is in the form of a braid embedded within the material of the sleeve.

18. An apparatus according to claim 15, wherein one or both of the intermediate sleeve and the elongated tube is provided with friction reducing features, the friction reducing features being a plurality of projections, and the wire embedded within the material of the sleeve aligns with and supports the plurality of projections.

19. An apparatus according to claim 1, wherein the intermediate sleeve is flexible.
